(19) United States
(12) Patent Application Publication
(10) Pub. No.: US 2025/0161696 A1
(43) Pub. Date: May 22, 2025

(54) RECHARGING IMPLANTABLE MEDICAL DEVICES

(71) Applicant: Cardiac Pacemakers, Inc., St Paul, MN (US)

(72) Inventors: Keith R. Maile, New Brighton, MN (US); Ignacio Chi, St Paul, MN (US); Joey Chen, Stevenson Ranch, CA (US); Jean M. Bobgan, Maple Grove, MN (US)

(21) Appl. No.: 18/952,997

(22) Filed: Nov. 19, 2024

Related U.S. Application Data
(60) Provisional application No. 63/600,954, filed on Nov. 20, 2023.

(51) Int. Cl. A61N 1/378

(52) U.S. Cl. CPC A61N 1/3787

(57) ABSTRACT

A system includes a medical device that is implantable. The medical device includes circuitry for processing physiological data, a battery that is rechargeable and that is electrically coupled to the circuitry to provide power to the circuitry, a charge coil arranged to generate current responsive to externally-generated magnetic fields and electrically coupled to the battery to charge the battery, and a magnetic field guide arranged to guide the externally-generated magnetic fields towards the charge coil.

### CROSS REFERENCE TO RELATED APPLICATION

This application claims priority to Provisional Application No. 63/600,954, filed Nov. 20, 2023, which is herein incorporated by reference in its entirety.

### TECHNICAL FIELD

Instances of the present disclosure relate to medical devices and systems for sensing physiological parameters and/or delivering therapy. In particular, instances of the present disclosure feature approaches for charging medical devices that are implantable.

### BACKGROUND

Implantable medical devices can be powered by a battery. However, to conserve space, the battery may be relatively small, have limited energy-storage capacity, and need to be recharged periodically.

### SUMMARY

In Example 1, a system includes a medical device that is implantable. The medical device includes circuitry for processing physiological data, a battery that is rechargeable and that is electrically coupled to the circuitry to provide power to the circuitry, a charge coil arranged to generate current responsive to externally-generated magnetic fields and electrically coupled to the battery to charge the battery, and a magnetic field guide arranged to guide the externally-generated magnetic fields towards the charge coil.

In Example 2, the system of Example 1, wherein the magnetic field guide is a sheet or a strip positioned between the battery and the charge coil.

In Example 3, the system of Example 2, wherein the charge coil is positioned adjacent to and coupled to a major surface of the battery.

In Example 4, the system of any of Examples 1-3, wherein the charge coil has a first thickness, wherein the magnetic field guide has a second thickness, wherein the first thickness and the second thickness are 100 μm or less.

In Example 5, the system of any of Examples 1-4, wherein the charge coil is positioned external to an outer housing of the medical device.

In Example 6, the system of Example 1, wherein the magnetic field guide is a cylinder of material positioned within the charge coil.

In Example 7, the system of any of Examples 1-6, wherein the magnetic field guide comprises ferrite.

In Example 8, the system of any of Examples 1-7, wherein a maximum of 5 milliwatts of power is used for charging the battery.

In Example 9, the system of Example 1, wherein the medical device includes a housing, wherein a first section of the housing comprises a ceramic material that creates and surrounds an internal cavity.

In Example 10, the system of Example 9, wherein the charge coil and the magnetic field guide are positioned within the internal cavity.

In Example 11, the system of Example 9, wherein the medical device includes an antenna positioned within the internal cavity.

In Example 12, the system of any of Examples 1-11, further including a power transmitter including a power transmitting coil configured to generate the externally-generated magnetic fields.

In Example 13, the system of Example 12, wherein the power transmitter further includes circuitry programmed to determine that the medical device is within range of the power transmitter and, in response, to turn on the power transmitting coil to generate the externally-generated magnetic fields.

In Example 14, the system of any of Examples 12 and 13, wherein the circuitry is further programmed to determine proximity of an object other than the medical device and, in response, reduce an amount of power applied to the power transmitting coil.

In Example 15, the system of Example 12, wherein the power transmitter further includes a cooling unit.

In Example 16, a system includes a medical device that is implantable. The medical device includes a battery that is rechargeable and that is electrically coupled to electronic components to provide power to the electronic components, a charge coil arranged to generate current responsive to externally-generated magnetic fields and electrically coupled to the battery to charge the battery, and a magnetic field guide arranged to guide the externally-generated magnetic fields towards the charge coil.

In Example 17, the system of Example 16, wherein the magnetic field guide is a sheet or a strip positioned between the battery and the charge coil.

In Example 18, the system of Example 17, wherein the charge coil is positioned adjacent to and coupled to a major surface of the battery.

In Example 19, the system of Example 17, wherein the charge coil has a first thickness, wherein the magnetic field guide has a second thickness, wherein the first thickness and the second thickness are 100 μm or less.

In Example 20, the system of Example 17, wherein the battery is positioned within an outer housing of the medical device, wherein the charge coil and the magnetic field guide are positioned external to the outer housing.

In Example 21, the system of Example 17, wherein the magnetic field guide comprises ferrite.

In Example 22, the system of Example 16, wherein the magnetic field guide is a cylinder of material positioned within the charge coil.

In Example 23, the system of Example 22, wherein the magnetic field guide comprises ferrite.

In Example 24, the system of Example 22, wherein a center axis of the cylinder is parallel to a center axis of the charge coil.

In Example 25, the system of Example 22, wherein a center axis of the charge coil is perpendicular to a longitudinal axis of the medical device.

In Example 26, the system of Example 16, wherein the medical device includes a housing, wherein a first section of the housing comprises a ceramic material that creates and surrounds an internal cavity.

In Example 27, the system of Example 26, wherein the charge coil and the magnetic field guide are positioned within the internal cavity.

In Example 28, the system of Example 16, further including a power transmitter with a power transmitting coil and circuitry. The power transmitting coil is configured to generate the externally-generated magnetic fields. The circuitry is configured to determine that the medical device is within range of the power transmitter and, in response, to turn on the power transmitting coil to generate the externally-generated magnetic fields.

In Example 29, a power transmitter including a housing, one or more proximity sensors configured to generate a output signal indicative of proximity of a medical device to the power transmitter, a power transmitting coil configured to generate magnetic fields for transmission external to the housing, and circuitry. The circuitry is programmed to determine, based at least on part on the output signal, that the medical device is within range of the power transmitter and, in response, to turn on the power transmitting coil to generate the magnetic fields.

In Example 30, the power transmitter of Example 29, wherein the circuitry is further programmed to determine that an object other than the medical device is within range of the power transmitter and, in response, reduce or turn off an amount of power applied to the power transmitting coil.

In Example 31, the power transmitter of Example 29, wherein the circuitry is further programmed to determine that a battery of the medical device has a charge level lower than a threshold and, in response, to turn on the power transmitting coil.

In Example 32, the power transmitter of Example 29, wherein the output signal is a signal strength indicator.

In Example 33, a method includes determining that a medical device is within a first range from the power transmitter; applying power to a power coil of a power transmitter after the determining that the medical device is within the first range; generating magnetic fields by the power coil to recharge a battery of the medical device; determining that an object other than the medical device is within a second range from the power transmitter; and reducing or turning off power applied to the power coil after the determining that the object is within the second range.

In Example 34, the method of Example 33, wherein the determining that the medical device is within the first range and the determining that the object is within the second range are both based, at least in part, on an output signal from one or more proximity sensors.

In Example 35, the method of Example 33, further including transmitting the magnetic fields towards a magnetic field guide positioned adjacent to a recharging coil positioned in the medical device.

In Example 36, an implantable medical device includes a battery that is rechargeable and that is electrically coupled to electronic components to provide power to the electronic components. The implantable medical device further includes a charge coil arranged to generate current responsive to externally-generated magnetic fields. The charge coil is wrapped around an outer housing of the implantable medical device.

In Example 37, the implantable medical device of Example 36, wherein the outer housing includes an electrical via that is electrically positioned between the charge coil and the battery.

In Example 38, the implantable medical device of Examples 36 and 37, wherein the charge coil is electrically coupled to the battery to charge the battery.

In Example 39, the implantable medical device of Examples 36-38, wherein the charge coil is wrapped around an outer circumference of the outer housing.

In Example 40, the implantable medical device of Examples 36-39, wherein the charge coil is only wrapped around the battery portion of the outer housing.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration depicting a medical device and charging system, in accordance with certain instances of the present disclosure.

FIGS. 2-4 show different views of a medical device, in accordance with certain instances of the present disclosure.

FIGS. 5 and 6 show different configurations of a charge coil, in accordance with certain instances of the present disclosure.

FIGS. 7-9 show different configurations of a magnetic field guide for use with medical devices, in accordance with certain instances of the present disclosure.

FIG. 10 shows a transmitter, in accordance with certain instances of the present disclosure.

FIG. 11 shows a block diagram depicting an illustrative method, in accordance with certain instances of the disclosure.

FIGS. 12 and 13 show different configurations of a charge coil, in accordance with certain instances of the present disclosure.

FIG. 14 is a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Medical devices can utilize rechargeable batteries to power various electrical components. For medical devices that are implanted, the process of recharging the batteries can be inconvenient for patients. Adding to the inconvenience is the need to track and remember when to recharge the batteries so that the medical device remains operational.

Further, there is a tension between (1) wanting to increase the energy-storage capacity of batteries—which may involve physically larger batteries—to reduce how often recharging is needed and (2) wanting to reduce the overall size of medical devices—which may involve utilizing physically smaller batteries.

Certain instances of the present disclosure are directed to approaches that allow medical devices to be conveniently recharged using wireless power transmission. Such approaches may allow for physically smaller batteries to be utilized to allow for medical device designs with smaller form factors.

However, wirelessly recharging a battery in an implanted medical device introduces certain technical challenges. One challenge occurs when the implantable medical device comprises a conductive material, which can interfere with signals intended for recharging the battery. Certain instances of the present disclosure are therefore directed to approaches that can reduce the likelihood of interference caused by materials of implantable medical devices. In particular, certain instances involve medical devices that utilize magnetic field guides to help focus electromagnetic fields towards charging coils.

### Medical Device Charging System

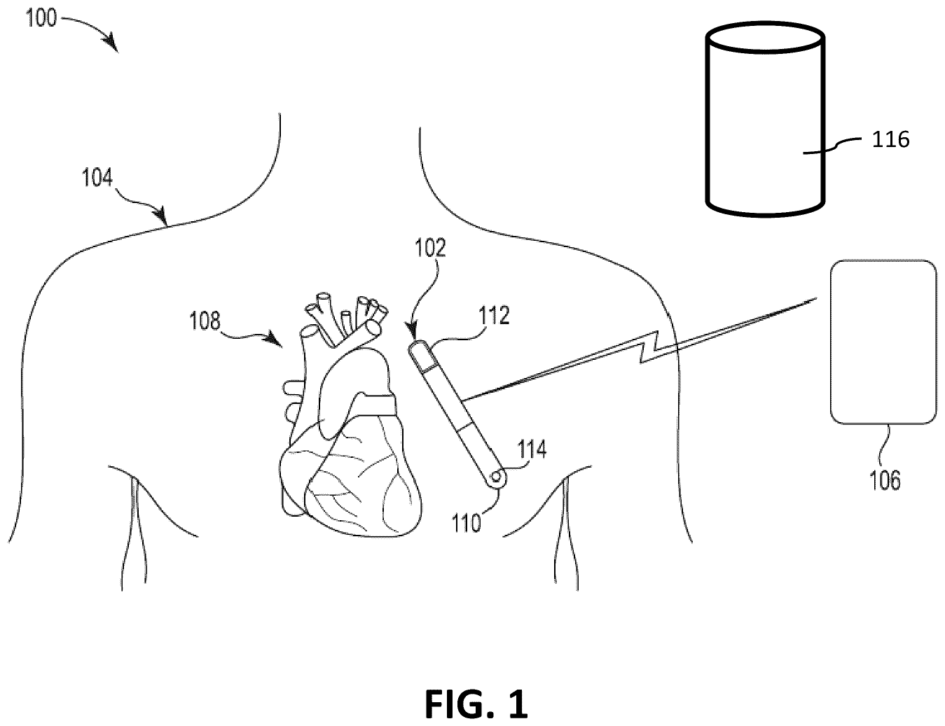

FIG. 1 is a schematic illustration of a system 100 including an implantable medical device (IMD) 102 implanted within a patient's body 104 and configured to communicate with a receiving device 106.

The IMD 102 may be implanted subcutaneously within an implantation location or pocket in the patient's chest or abdomen and may be configured to monitor (e.g., sense and/or record) physiological parameters associated with the patient's heart 108. The IMD 102 may be an implantable cardiac monitor (e.g., an implantable diagnostic monitor, an implantable loop recorder) configured to record physiological parameters such as, for example, one or more cardiac activation signals, heart sounds, blood pressure measurements, oxygen saturations. Further, the IMD 102 may be configured to monitor physiological parameters that may include one or more signals indicative of a patient's physical activity level and/or metabolic level, such as an acceleration signal.

For example, the IMD 102 may include a control device, a monitoring device, a pacemaker, an implantable cardioverter defibrillator (ICD), a cardiac resynchronization therapy (CRT) device and/or the like, and may be an implantable medical device known in the art or later developed, for providing therapy and/or diagnostic data about the patient's body. In various instances, the IMD 102 may include both defibrillation and pacing/CRT capabilities (e.g., a CRT-D device). In other instances, the IMD 102 is configured as a urological therapy device to deliver selective stimulation to, for example, the sacral nerves for treatment of urological disorders such as bladder control or erectile disorders (e.g., an IMD with an electromechanical actuation device). In other examples, the IMD 102 is configured as a gastrointestinal device to treat, for instance, gastroesophageal reflux disease (GERD). In other examples, the IMD 102 may be configured as a neurostimulation therapy device for pain management and the like. In still other examples, the IMD 102 may be a cardiac rhythm management device for sensing and stimulating cardiac tissue for treatment of cardiac arrhythmias such as bradycardia, tachycardia and for cardiac resynchronization therapy. In still other examples, the IMD 102 may be configured as a monitoring device only, with no therapeutic functionality, to monitor physiological parameters of a patient. In short, the present disclosure is not limited to any clinical application, and any implantable device that requires power to operate as intended.

The example IMD 102 shown in FIG. 1 includes a housing 110 having two electrodes 112 and 114 coupled thereto. The IMD 102 may be configured to sense physiological parameters and record the physiological parameters. For example, the IMD 102 may be configured to activate (e.g., periodically, continuously, upon detection of an event, and/or the like), record a specified amount of data (e.g., physiological parameters) in a memory, and communicate that recorded data to the receiving device 106. The recording device 106 may be, for example, a programmer, controller, patient monitoring system, and/or the like. Although illustrated in FIG. 1 as an external device, the receiving device 106 may include an implantable device configured to communicate with the IMD 102 that may, for example, be a control device, another monitoring device, a pacemaker, an ICD, a CRT device, and/or the like.

The IMD 102 and the receiving device 106 may communicate through a wireless link. For example, the IMD 102 can include an antenna, which transmits and/or receives signals from the receiving device 106. The IMD 102 and the receiving device 106 may be communicatively coupled through a short-range communications link, such as Bluetooth, IEEE 802.11, and/or a proprietary wireless protocol. The communications link may facilitate uni-directional and/or bi-directional communication between the IMD 102 and the receiving device 106. Data and/or control signals may be transmitted between the IMD 102 and the receiving device 106 to coordinate the functions of the IMD 102 and/or the receiving device 106. The patient data may be downloaded from one or more of the IMD 102 and the receiving device 106 periodically or on command. The physician and/or the patient may communicate with the IMD 102 and the receiving device 106, for example, to acquire patient data or to initiate, terminate, or modify recording and/or therapy.

The system 100 further includes a power transmitter 116. The power transmitter 116 is designed to generate and transmit magnetic fields, which can be used to recharge a battery of the IMD 102. Like with the receiving device 106, the IMD 102 and the power transmitter 116 may also communicate through a wireless link such as Bluetooth, IEEE 802.11, etc.

### Medical Device

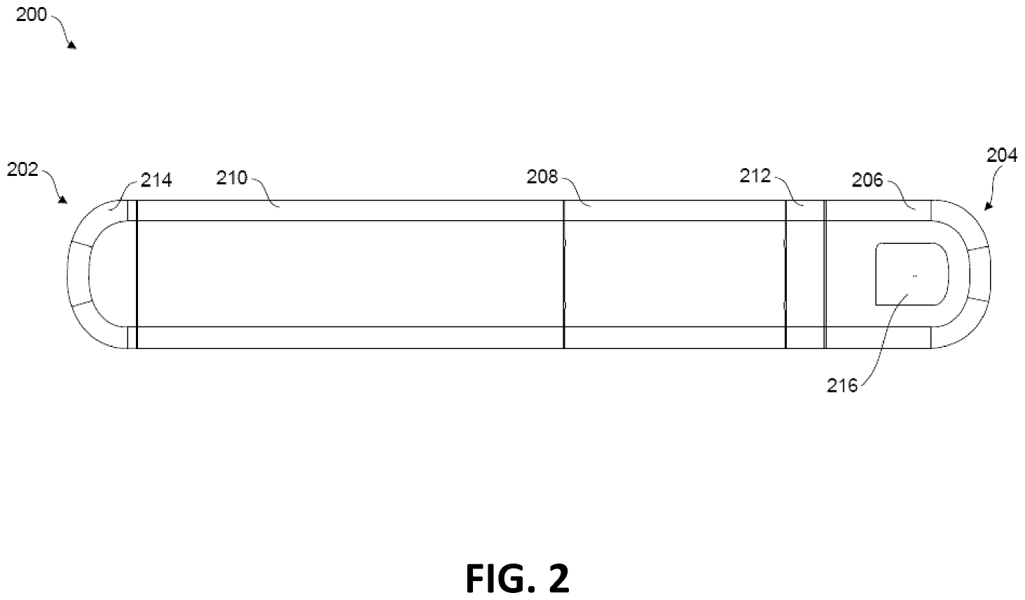

FIG. 2 is a side view of a medical device 200 (hereinafter “IMD 200” for brevity). The IMD 200 may be, or may be similar to, the IMD 102 depicted in FIG. 1 and may be used in the system 100 of FIG. 1.

The IMD 200 includes an external housing that extends between a first end 202 and a second end 204. In the example of FIG. 2, the IMD 200 includes a first housing section 206, a second housing section 208, a third housing section 210, a fourth housing section 212, a first electrode 214, and a second electrode 216. Each of the housing sections can be separate components that are assembled together during manufacturing to create the external housing of the IMD 200. When assembled together, the housing sections can create a hermetically sealed enclosure. Although four separate housing sections are shown in FIG. 2, additional or fewer separate sections can be used to create the IMD 200. As will be described in more detail below, one or more of the housing sections can comprise a ceramic material.

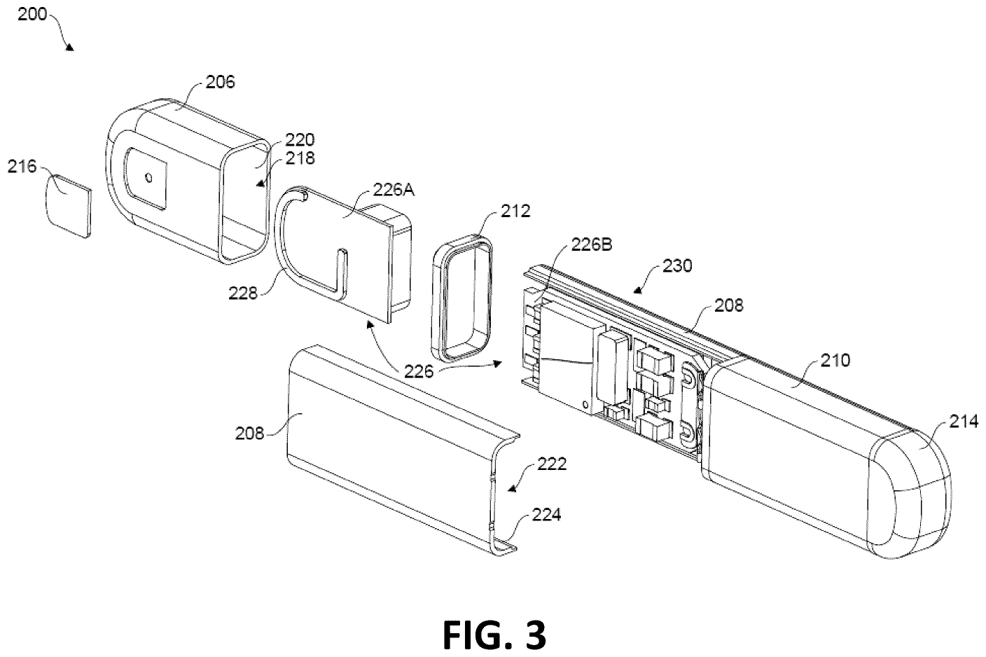

FIG. 3 shows a partially exploded view of the IMD 200. The first housing section 206 includes a first cavity 218 that is defined by one or more interior surfaces 220 of first housing section 206. The second electrode 216 is coupled to the first housing section 206. In certain instances, the first housing section 206 comprises a ceramic material.

The second housing section 208 includes a second cavity 222 that is defined by one or more interior surfaces 224 of second housing section 208. In FIG. 2, the second housing section 208 is shown as being comprised of multiple housing components. For example, the second housing section 208 (as with other housing sections) can be assembled from multiple components (e.g., welded together) to create its portion of the external housing of the IMD 200. In certain instances, the second housing section 208 comprises a metal material such as titanium. In other instances, the second housing section 208 comprises a ceramic material.

The third housing section 210 can comprise or can be a battery assembly (which may include one or more batteries). The exterior of the battery assembly can form the third housing section 210 in which one or more batteries (e.g., rechargeable battery cells) are positioned. The first electrode 214 is disposed at an end of the third housing section 210. In certain instances, the first electrode 214 is integrated with the battery assembly.

The fourth housing section 212 can function as an interface or coupler between the first housing section 206 and the second housing section 208. For example, the fourth housing section 212 can be used to couple the first housing section 206 to the second housing section 208. More specifically, in instances where the fourth housing section 212 comprises a metal such as titanium, the fourth housing section 212 can be assembled to the second housing section 208 via welding (e.g., laser welding). And, in instances where the first housing section 206 comprises a ceramic, the fourth housing section 212 can be brazed to the first housing section 206. In such instances, the fourth housing section 212 can be coupled between the first housing section 206 and the second housing section 208. As shown in FIG. 3, the fourth housing section 212 can be shaped as a continuous ring with an opening therethrough. Further, the fourth housing section 212 can include joint features such as one or more thinned sections or flange sections such that connecting the fourth housing section 212 to the other sections (e.g., via welding and/or brazing) can be accomplished. For example, portions of the other housing sections can overlap with the thinned or flanged sections of the fourth housing section 212 to provide overlapping surface area.

FIG. 3 shows a circuit board 226 with a first circuit board section 226A and a second circuit board section 226B. For purposes of illustrating the various components of the IMD 200 in an exploded view, the first circuit board section 226A and the second circuit board section 226B are shown as two separate components, but the two sections can form a single circuit board. The first circuit board section 226A and the second circuit board section 226B can comprise a rigid circuit board or a flexible circuit (e.g., a flexible circuit comprising polyimide). In instances where the first circuit board section 226A and the section circuit board section 226B are separate components, one section can comprise a rigid circuit board and the other section can comprise a flex circuit. Further, the two sections can be electrically coupled to each other.

When the IMD 200 is assembled, the first circuit board section 226A is positioned within the first cavity 218 and the second circuit board section 226B is positioned within the second cavity 222. Portions of either or both of the first circuit board section 226A and the second circuit board section 226B can extend through the fourth housing section 212 when the IMD 200 is assembled. As such, in instances with a single, continuous circuit board, the circuit board 226 can extend within the first cavity 218 and the second cavity 222 and through the opening of the fourth housing section 212.

An antenna 228 is positioned within the first cavity 218 and coupled to the circuit board 226 such as to the first circuit board section 226A. As one example, the antenna 228 can be embedded within the first circuit board section 226A. In this example, the antenna 228 can be formed by a conductive trace in the first circuit board section 226A. As another example, the antenna 228 can be formed on the interior surface 220 of the first housing section 206 and electrically coupled (e.g., directly coupled or indirectly coupled) to a conductive trace in the first circuit board section 226A. In this example, a portion of the interior surface 220 can be metalized if the interior surface 220 is otherwise a ceramic material. As another example, the antenna 228 can be embedded in the first housing section 206 and electrically coupled to a conductive trace in the first circuit board section 226A. In other instances, the antenna 228 can be positioned within the external housing between the third housing section and the first electrode 214 (e.g., positioned in a space between the battery and the first electrode 214). In other instances, the antenna 228 can be positioned outside the external housing.

Various other electrical components 230 can be coupled to the circuit board 226 such as on the second circuit board section 226B. The electrical components 230 can include memory along with one or more integrated circuits (e.g., application specific integrated circuits, field-programmable gate arrays) programmed to perform functions such as sensing, processing, and/or communication functions of the IMD 200. For example, the electrical components 230 can include one or more processors (e.g., microprocessors) coupled to memory with instructions (e.g., in the form of firmware, and/or software) for performing functions of the IMD 200. Example functions of the circuitry include processing physiological data (e.g., converting sensed electrical signals from the electrodes into electrocardiogram data, detecting potential cardiac events based on the sensed electrical signals, saving physiological data to the memory, and the like). The electrical components 230 can be electrically coupled to the one or more batteries such that the electrical components 230 are powered by the one or more batteries.

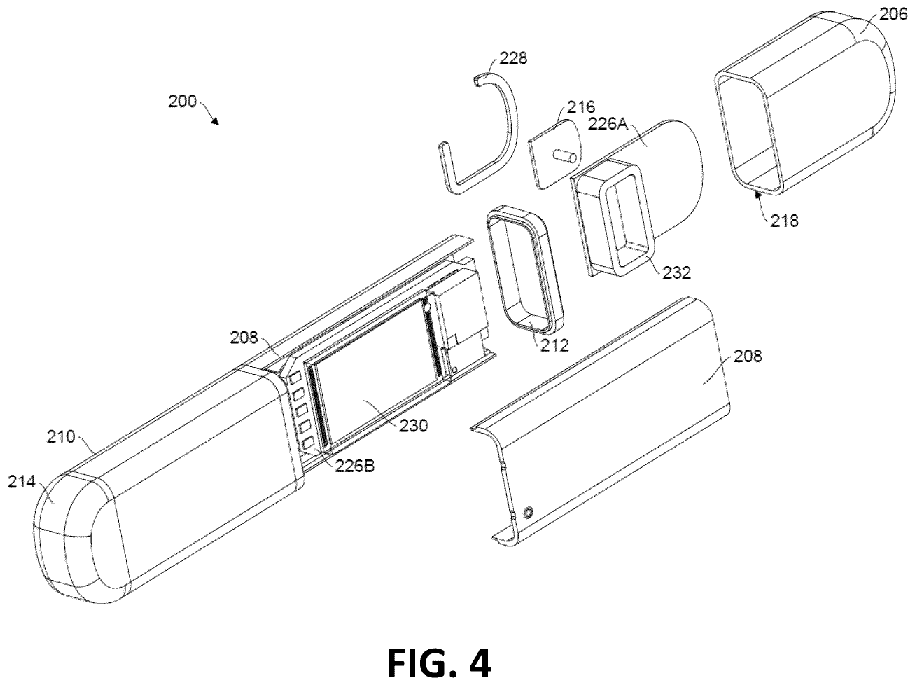

FIG. 4 shows another view of the IMD 200. As shown in FIG. 4, in certain instances, a charge coil 232 (e.g., a set of coils comprising a conductive material) is coupled to the circuit board 226 such as the first circuit board section 226A. The charge coil 232 is arranged to receive external signals (e.g., electromagnetic signals generated by a power transmitter) that induce a current in the charge coil 232 such that the current can recharge batteries in the battery assembly. When the IMD 200 is assembled, the charge coil 232 is positioned within the first cavity 218 of the first housing section 206 such that electromagnetic signals can pass through the housing and reach the charge coil 232. In certain instances, the charge coil 232 is wound such that there is a hole or space through the center of the charge coil 232 (e.g., the charge coil 232 has a donut shape or a similar shape). The charge coil 232 is electrically coupled to the one or more batteries of the IMD 200. In certain instances, a magnetic field guide is coupled between the charge coil 232 and the circuit board 226 such that the magnetic field guide is directly coupled to the circuit board 226. Further details of magnetic field guides are discussed herein.

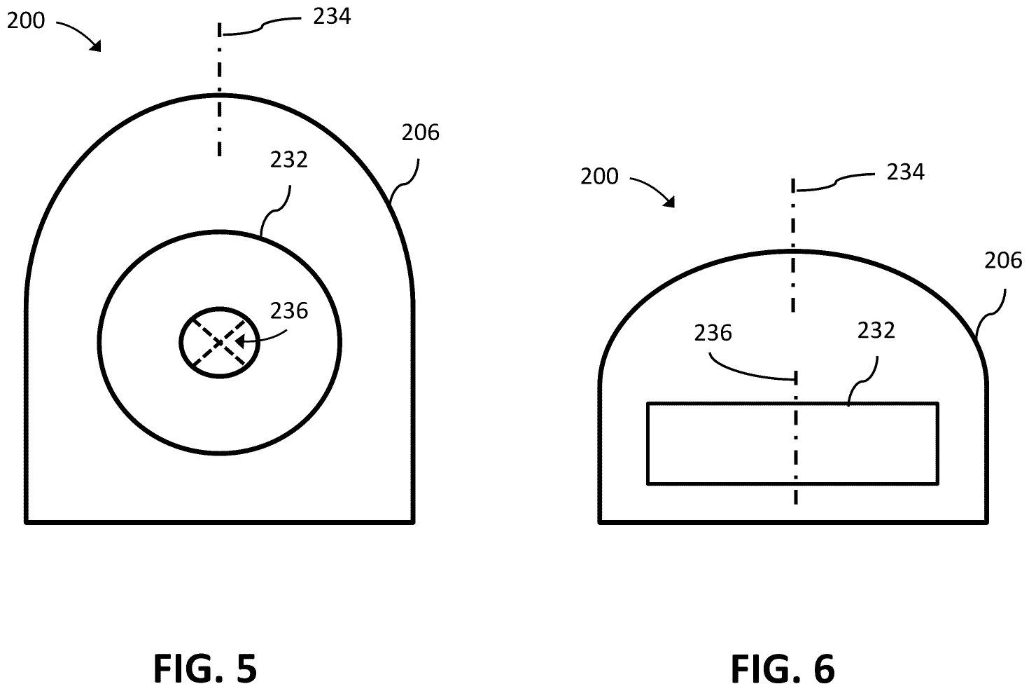

FIGS. 5 and 6 show simplified schematics of the charge coil 232 in different orientations relative to a longitudinal axis 234 of the IMD 200.

In FIG. 5, the charge coil 232 is positioned in the first housing section 206 and includes a center axis 236 (e.g., an axis into and out of the page through the center of the “X” shown in FIG. 5). The charge coil 232 is oriented such that the center axis 236 of the charge coil 232 is perpendicular to the longitudinal axis 234 of the IMD 200. As such, the diameter or length of the charge coil 232 along the longitudinal axis 234 may be greater than the thickness of the charge coil 232 along the direction of the center axis 236.

In FIG. 6, the charge coil 232 is oriented such that the center axis 236 of the charge coil 232 is parallel to the longitudinal axis 234 of the IMD 200. In this orientation, the charge coil 232 consumes less space along the longitudinal axis 234 (compared to the orientation in FIG. 5) which can allow the overall length of the IMD 200 to be reduced.

### Magnetic Field Guides

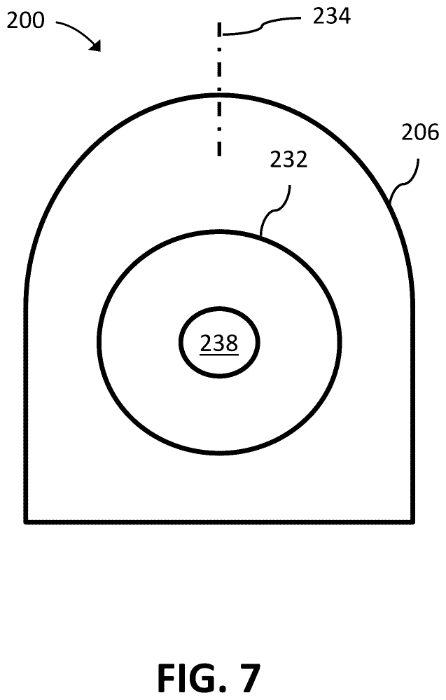

FIGS. 7-9 show different configurations of magnetic field guides, which help focus externally-generated electromagnetic fields towards the charge coil 232 of the IMD 200. The IMD 200 can comprise a conductive material as part of the external housing or other parts, and that conductive material can interfere with the electromagnetic fields intended for recharging the battery via the charge coil 232. The magnetic field guides can be designed and arranged to help reduce the likelihood of interference caused by a conductive material of the IMD 200.

FIG. 7 shows a magnetic field guide 238 positioned within the charge coil 232 such that the charge coil 232 at least partially surrounds the magnetic field guide 238. In certain instances, the magnetic field guide 238 is a cylinder (e.g., a solid cylinder) of a magnetic material (e.g., a magnetic material comprising iron). For example, the magnetic field guide 238 can be comprised of a ferrite material. In such examples, the magnetic field guide 238 can be considered to be a ferrite core or a ferrite slug that is positioned within the charge coil 232. In other instances, the magnetic field guide 238 comprises a metamaterial.

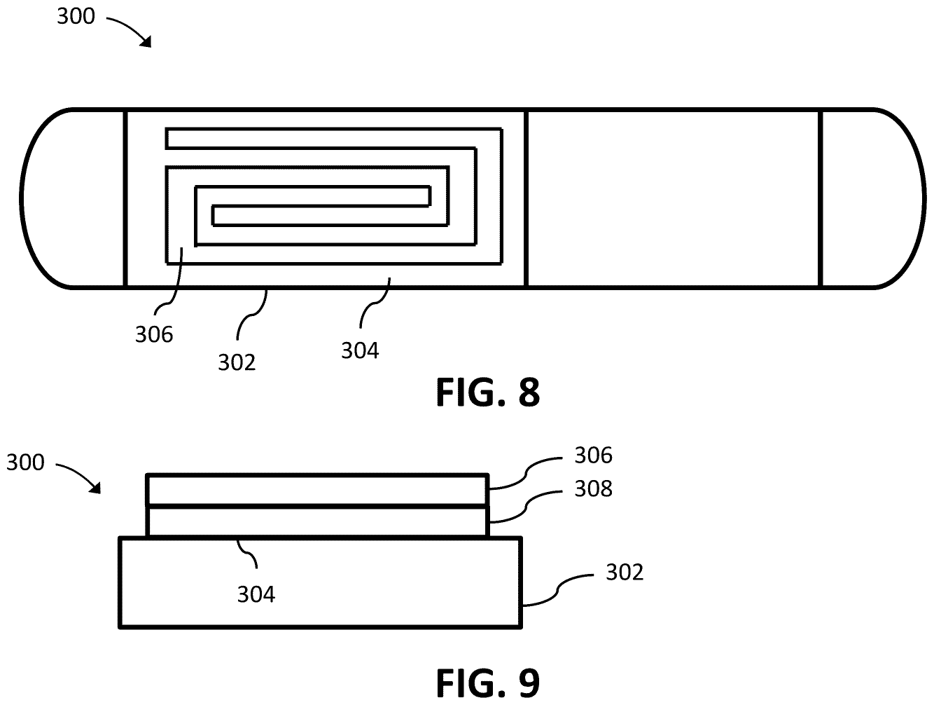

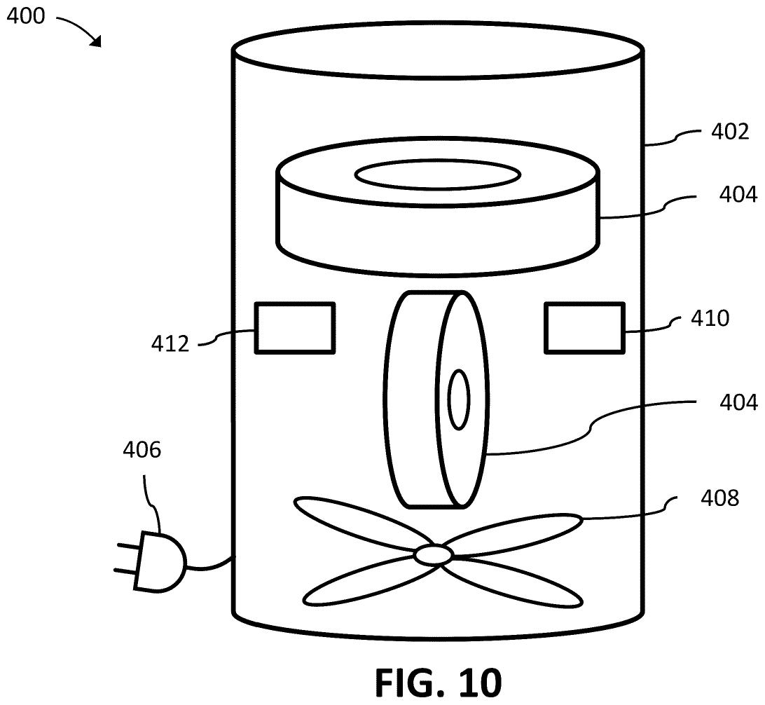

FIG. 8 shows a top view of an IMD 300 with a different configuration of a magnetic field guide, and FIG. 9 shows a side view of a portion of the IMD 300. For simplicity of illustration, the IMD 300 of FIGS. 8 and 9 is shown in a simplified schematic with fewer details than the IMD 200 shown in prior figures. However, the IMD 300 of FIGS. 8 and 9 can include similar features to the IMD 200 (e.g., electrodes, circuitry, ceramic housing sections, and other features discussed above). In particular, the magnetic field guide and charging coil arrangement of FIGS. 8 and 9 can be used in connection with features of the IMD 200 and in place of the charging coil 232 and magnetic field guide 238.

Referring to FIG. 8, the IMD 300 includes a battery 302 or battery assembly that includes one or more battery cells (e.g., rechargeable battery cells such as battery cells that comprise lithium). The battery 302 can include an outer housing that surrounds and protects the battery cells. The battery 302 can be substantially cube-shaped and have six outer surfaces, including two major surfaces 304 which have the largest surface area among the outer surfaces of the battery 302.

The IMD 300 also includes a charge coil 306 mechanically coupled (e.g., mechanically indirectly coupled) to the battery 302 (e.g., to the outer housing of the battery 302). In certain instances, the charge coil 304 is positioned along the major surface 304 of the battery 302. In this position, the overall length of the IMD 300 does not need to accommodate the charge coil 304. Put another way, because the charge coil 306 is not positioned within one of the other housing sections of the IMD 300, the overall length of the IMD 300 can be shorter than if the charge coil 306 was positioned similar to the position of the charge coil 232 of the IMD 200 of FIGS. 2-7. Further, the position of the charge coil 306 of FIG. 9 allows the overall size (e.g., length) of the charge coil 306 to be greater compared to other arrangements. Increasing the size of a charge coil can increase the efficiency of the charge coil. The specific shape of the charge coil 304 can vary depending on the geometry of the IMD 300, among other design constraints and considerations. For example, instead of the spiral shape shown in FIG. 8, the charge coil 306 could be substantially donut-shaped like the charge coils in prior Figures.

The charge coil 306 can be electrically coupled to the battery 302 such that current generated by the charge coil 306—in response to externally-generated electromagnetic fields—can be used to recharge the battery 302 (e.g., the battery cells of the battery 302).

FIG. 9 shows a side view of the battery portion of the IMD 300. A magnetic field guide 308 is shown being positioned between the battery 302 and the charge coil 306. The magnetic field guide 308 can be a sheet or a strip of magnetic material (e.g., the same materials as the magnetic field guide 238) positioned between the battery 302 and the charge coil 306. The magnetic field guide 308 can be a rectangular sheet that is coupled to the battery 302 or can be shaped to substantially match the shape of the charge coil 306. In certain instances, the magnetic field guide 308 and the charge coil 306 each have a thickness of less than 100 μm—and therefore have a combined thickness of no more than 200 μm. The magnetic field guide 308 and the charge coil 306 can be adhered together and coupled to the battery 302.

Other magnetic field guide arrangements can be used with the IMDs described herein. For example, a magnetic field guide similar to the magnetic field guide of FIGS. 8 and 9 could be coupled to a circuit board between the circuit board and a charging coil (e.g., the charging coil 232 of FIG. 4). In this arrangement, the charging coil (and magnetic field guide) could be positioned on one side of the circuit board while other electrical components are positioned on an opposite side of the circuit board. This arrangement could allow the overall length of the IMD to be shorter compared to the arrangements of FIGS. 5-7.

Additionally or alternatively, a strip of ferrite (or similar magnetic material) could be positioned within parts of a charging coil.

Regardless of the type of magnetic field guide used, the charge coil can be designed to have a resonant frequency within ISM Bands (industrial-, scientific-, or medical-frequency bands). In certain instances, the charge coil 234 is designed to be a 6.78 MHz resonant coil or a 13.56 MHz resonant coil. Further, the charge coil 234 can be tuned (e.g., by selection of the number of coil turns) for low power charging, which is discussed in more detail below with respect to example power transmitters. As one specific example, the charge coil 234 can generate no more than 5 milliwatts (mW) of power for charging the one or more batteries of the IMD 200. Utilizing a low power (e.g., 5 mW or less) can help reduce the risks of overheating or shorting the one or more batteries (e.g., by using a low charging current). For example, using a low charging current (e.g., approximately 1 mA or less) can reduce the risk of thermal runaway in the event the lithium metal in the battery becomes plated to an anode of the battery.

### Power Transmitter

FIG. 9 shows a schematic of a power transmitter 400 (hereinafter the “transmitter 400” for brevity). The transmitter 400 can be positioned such that a patient with an IMD can be at a location that is convenient for charging the one or more batteries of the IMD. For example, the transmitter 400 can be positioned such that the patient can be sitting in a chair or lying down on a bed while the transmitter 400 generates power to recharge the one or more batteries. Example positions of the transmitter 400 include on a piece of furniture (e.g., a nightstand, a dresser, a table, and the like), coupled to a wall or ceiling, or positioned within any of the preceding examples. As such, the transmitter 400 can be portable or designed for more permanent installations. In certain instances, the transmitter 400 is designed to transmit up to 5 mW of power within one meter of the transmitter 400. However, it is noted that the amount of power varies (e.g., decreases) as the distance away from the transmitter 400 increases.

The transmitter 400 includes a housing 402, one or more power-generating or power-transmitting coils 404, a power input 406, one or more cooling units 408, one or more sensors 410, and circuitry 412.

The housing 402 can be shaped to fit on or within the various locations mentioned above. For example, the housing 402 can be shaped to sit on furniture or to be coupled to a wall or ceiling.

The coil 404 can be designed and controlled to generate an electromagnetic field at one or more frequencies that match the resonant frequency of a charging coil of an IMD. For example, the coil 404 can be designed as a 6.78 MHz resonant coil or a 13.56 MHz resonant coil. In certain instances, the transmitter 400 can include more than one coil. For example, the coils can be arranged at different orientations such that the transmitter 400 can transmit electromagnetic fields in multiple directions such that the patient does not need to be concerned with their own orientation and location with the range of the transmitter 400.

As mentioned above, the charge coils of the IMDs described above may be designed to generate no more than 5 mW of power for charging the one or more batteries. With the 5-mW constraint and a goal of transmitting power up to 1 meter, the coil 404 may require approximately 100 Watts of input power to meet such constraints and goals. To generate such power, the transmitter 400 can be coupled to an external power source via a power input 406. For example, the power input 406 can be plugged into wall electricity to receive the power needed to generate the electromagnetic fields and operate other electronic components of the transmitter 400.

Because the coil 404 not only generates an electromagnetic field but also generates heat during operation, the transmitter 400 can include one or more cooling units 408 to cool the interior of the transmitter 400. The cooling unit 408 can comprise an air mover such as a fan, blower, and the like.

The one or more sensors 410 can include a proximity sensor (e.g., a capacitance sensor, a photoelectric sensor, a Bluetooth sensor), a temperature sensor (e.g., a thermistor, a thermocouple), and/or a magnetic field sensor (e.g., a Hall sensor, magnetoresistive sensor) among other types of sensors. The one or more sensors 410 can generate output signals and communicate the output signals to the circuitry 412.

The circuitry 412 can be programmed to control various electronic components of the transmitter 400.

As one example, the circuitry 412 can receive output signals from a temperature sensor and—based on the circuitry's programming—determine to power (and how much power to apply to) the cooling unit 408 in response to a detected temperature.

As another example, the circuitry 412 can receive output signals from a proximity sensor and determine that a person, animal, object, etc., is close to the transmitter 400. If too close, the circuitry 412 could reduce or turn off power to the coil 404 to limit exposure to electromagnetic fields.

As another example, the circuitry 412 can receive output signals from a proximity sensor and determine whether an IMD is within range (e.g., 1 meter) of the transmitter 400. More specifically, if the IMD and the transmitter 400 communicate via a Bluetooth wireless link, the circuitry 412 can use Bluetooth protocol (e.g., Bluetooth's “Received Signal Strength Indicator”) to detect when the IMD is within range of the transmitter 400. This approach can also be used by the circuitry 412 to control the amount of power applied to the coil 404 such that less power is used as the IMD becomes closer positioned to the transmitter 400. In certain instances, the amount of power applied to the coil 404 is controlled such that the battery of the IMD is trickle-charged (e.g., gradually recharged while the patient is sleeping). For example, if the target maximum charging power for the IMD is ˜5 mW, charging for 8 hours per day results in 40 mWh of charging per day.

As another example, the circuitry 412 can receive data (e.g., status information) from the IMD about its battery charge level and capacity and determine whether to recharge the battery using the transmitter 400.

### Methods

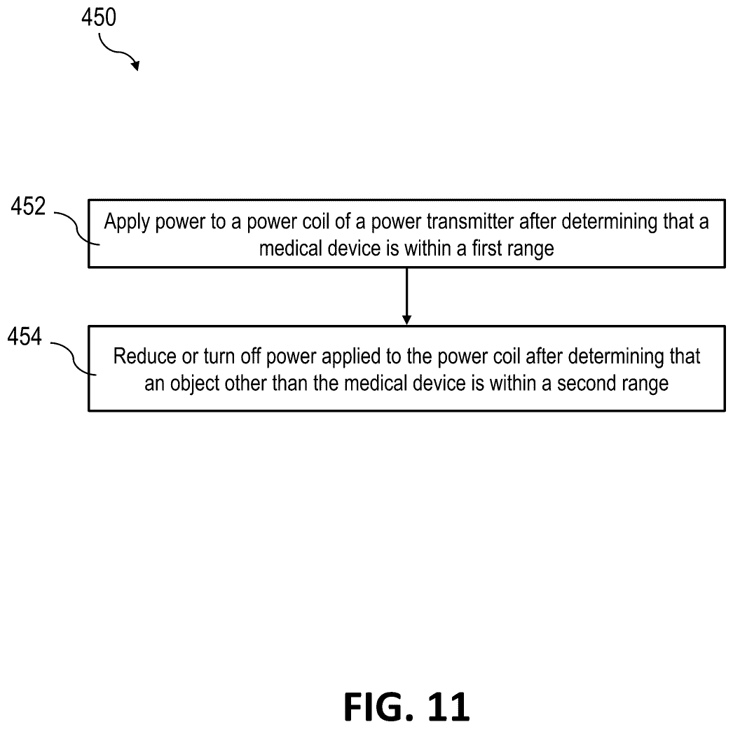

FIG. 11 shows an outline of steps of a method 450 for operating a power transmitter to wirelessly recharge a battery of a medical device and reducing the risk of exposing other objects to magnetic fields. The method 450 can include various steps and/or functions described above and is not limited necessarily to the steps shown in FIG. 11.

The method 450 includes applying power to a power coil of a power transmitter after determining that a medical device is within a first range (block 452 of FIG. 11). The method 450 further includes reducing or turning off power applied to the power coil after determining that an object other than the medical device is within a second range (block 454 of FIG. 11).

### Additional Charge Coil Approaches

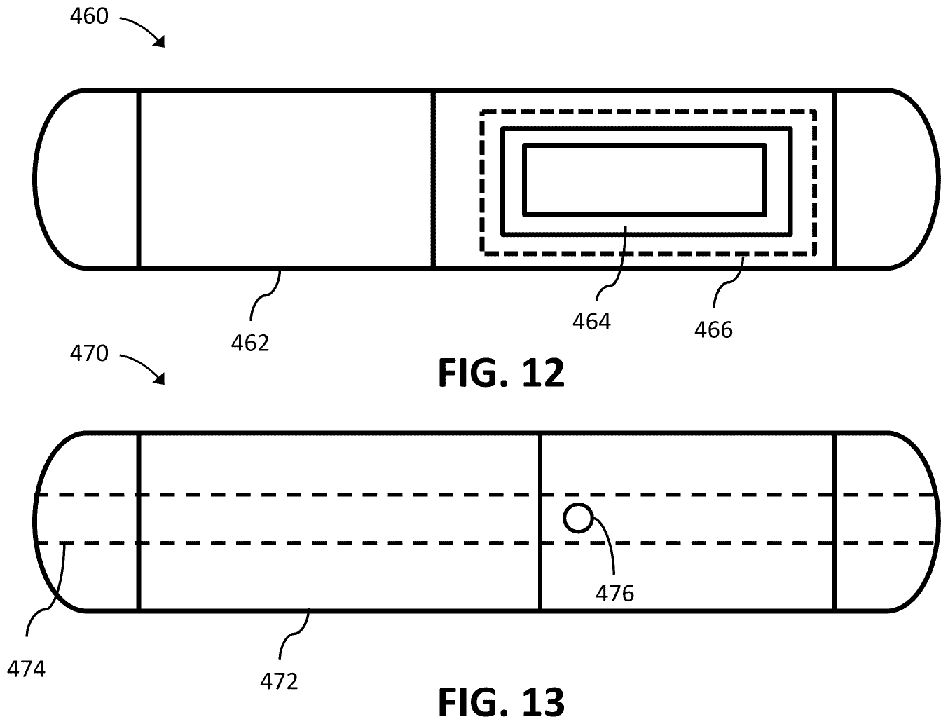

FIGS. 12 and 13 show additional approaches for positioning charge coils within or outside an IMD. For simplicity of illustration, the IMDs of FIGS. 12 and 13 are shown in a simplified schematic with fewer details than the IMD 200 shown in prior figures. However, the IMDs of FIGS. 12 and 13 can include similar features to the IMD 200 (e.g., electrodes, circuitry, ceramic housing sections, and other features discussed above).

FIG. 12 shows a top view of an IMD 460, which includes a battery 462 or battery assembly that includes one or more battery cells (e.g., rechargeable battery cells such as battery cells that comprise lithium). The battery 462 can include an outer housing that surrounds and protects the battery cells.

The IMD 460 also includes a charge coil 464 positioned within a cavity of the IMD 460. The charge coil 464 can be mechanically coupled to a printed circuit board 466 (shown in dotted lines). In certain instances, the charge coil 464 is a flat, wound coil comprising a conductive material. However, the specific shape of the charge coil 464 (e.g., rectangular, square, circular, oval) can vary. The charge coil 464 can be electrically coupled to the battery 462 (e.g., via a conductive trace in the printed circuit board 466) such that current generated by the charge coil 464—in response to externally-generated electromagnetic fields—can be used to recharge the battery 462 (e.g., the battery cells of the battery 462).

FIG. 13 shows a top view of an IMD 470, which includes a battery 472 or battery assembly that includes one or more battery cells. The battery 472 can include an outer housing that surrounds and protects the battery cells.

The IMD 470 also includes a charge coil 474 (shown in dotted lines) attached to an exterior surface of the IMD 470. The charge coil 474 can be wrapped around the exterior surface of the IMD 470. In certain instances, the charge coil 474 is wrapped around an entire outer circumference of the IMD 470. In certain instances, the charge coil 474 is only wrapped around the battery 472 portion of the exterior surface of the IMD 470 such that the charge coil 474 does not interfere with antenna communications or electrodes. For example, the charge coil 474 could be wrapped or coiled around a substantial portion of the exterior surface of the battery 472.

By positioning the charge coil 474 outside the IMD 470, the dimensions of the interior (e.g., interior cavity) of the IMD 470 do not need to accommodate the charge coil 474. Further, the charge coil 474 may be able to be made larger compared to other arrangements. Increasing the size of a charge coil can increase the efficiency of the charge coil. The specific shape of the charge coil 474 can vary depending on the geometry of the IMD 474, among other design constraints and considerations.

Because the charge coil 474 is positioned external to the IMD 470, the charge coil 474 may be exposed to tissue, blood, etc., of the patient. As such, the charge coil 474 (or at least an exterior surface of the charge coil 474) can comprise a biocompatible material. For example, the charge coil 474 can comprises a biocompatible wire or conductor.

Further, because the charge coil 474 is positioned external to the IMD 470 and the battery 472 is positioned within the IMD 470, an electrical via 476 or feedthrough can be incorporated into the housing of the IMD 470. The electrical via 476 or feedthrough can be incorporated to provide a hermetic seal while still enabling electrical signals to be passed from the exterior to the interior of the IMD 470. As such, the charge coil 474 can be electrically coupled to the battery 472 so that current generated by the charge coil 474—in response to externally-generated electromagnetic fields—can be used to recharge the battery 472.

### Computing Devices and Systems

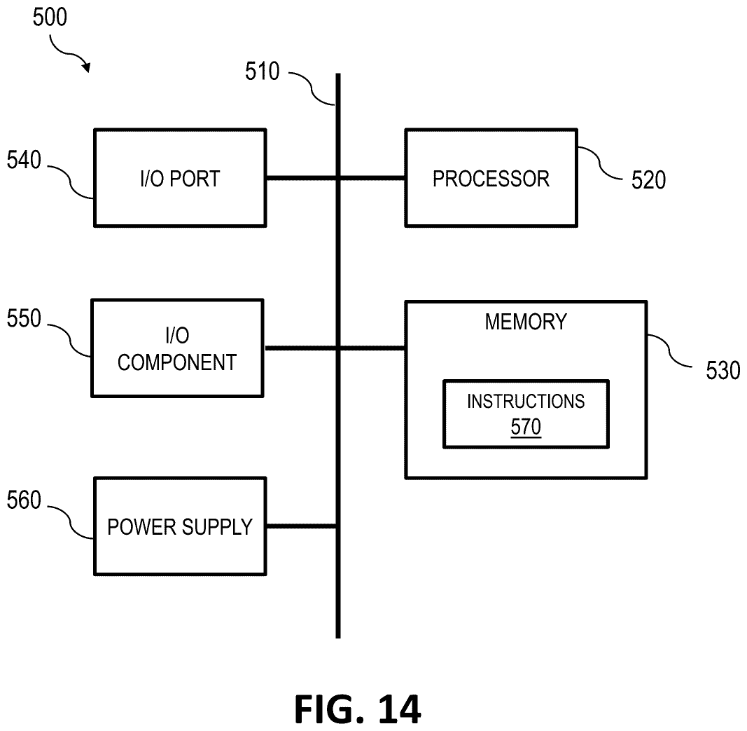

FIG. 14 is a block diagram depicting an illustrative computing device 500, in accordance with instances of the disclosure. The computing device 500 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 14 and described below. For example, IMDs and power transmitters can each include their own set (or partial set) of components shown in FIG. 14 and described below.

In instances, the computing device 500 includes a bus 510 that, directly and/or indirectly, couples one or more of the following devices: a processor 520, a memory 530, an input/output (I/O) port 540, an I/O component 550, and a power supply 560. Any number of additional components, different components, and/or combinations of components may also be included in the computing device 500.

The bus 510 represents what may be one or more busses (such as, for example, an address bus, data bus, or combination thereof). Similarly, in instances, the computing device 500 may include a number of processors 520, a number of memory components 530, a number of I/O ports 540, a number of I/O components 550, and/or a number of power supplies 560. Additionally, any number of these components, or combinations thereof, may be distributed and/or duplicated across a number of computing devices.

In instances, the memory 530 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 530 stores computer-executable instructions 570 for causing the processor 520 to implement aspects of instances of components discussed herein and/or to perform aspects of instances of methods and procedures discussed herein. The memory 530 can comprise a non-transitory computer readable medium storin the computer-executable instructions 570.

The computer-executable instructions 570 may include, for example, computer code, machine-useable instructions, and the like such as, for example, program components capable of being executed by one or more processors 520 (e.g., microprocessors) associated with the computing device 500. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 570 may be configured to be executed by the processor 520 and, upon execution, to cause the processor 520 to perform certain processes. In certain instances, the processor 520, memory 530, and instructions 570 are part of a controller such as an application specific integrated circuit (ASIC), field-programmable gate array (FPGA), and/or the like. Such devices can be used to carry out the functions and steps described herein.

The I/O component 550 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a medical device that is implantable and that includes:
a battery that is rechargeable and that is electrically coupled to electronic components to provide power to the electronic components,
a charge coil arranged to generate current responsive to externally-generated magnetic fields and electrically coupled to the battery to charge the battery, and
a magnetic field guide arranged to guide the externally-generated magnetic fields towards the charge coil.

2. The system of claim 1, wherein the magnetic field guide is a sheet or a strip positioned between the battery and the charge coil.

3. The system of claim 2, wherein the charge coil is positioned adjacent to and coupled to a major surface of the battery.

4. The system of claim 2, wherein the charge coil has a first thickness, wherein the magnetic field guide has a second thickness, wherein the first thickness and the second thickness are 100 μm or less.

5. The system of claim 2, wherein the battery is positioned within an outer housing of the medical device, wherein the charge coil and the magnetic field guide are positioned external to the outer housing.

6. The system of claim 2, wherein the magnetic field guide comprises ferrite.

7. The system of claim 1, wherein the magnetic field guide is a cylinder of material positioned within the charge coil.

8. The system of claim 7, wherein the magnetic field guide comprises ferrite.

9. The system of claim 7, wherein a center axis of the cylinder is parallel to a center axis of the charge coil.

10. The system of claim 7, wherein a center axis of the charge coil is perpendicular to a longitudinal axis of the medical device.

11. The system of claim 1, wherein the medical device includes a housing, wherein a first section of the housing comprises a ceramic material that creates and surrounds an internal cavity.

12. The system of claim 11, wherein the charge coil and the magnetic field guide are positioned within the internal cavity.

13. The system of claim 1, further comprising:
a power transmitter including a power transmitting coil and circuitry, wherein the power transmitting coil is configured to generate the externally-generated magnetic fields, wherein the circuitry is configured to determine that the medical device is within range of the power transmitter and, in response, to turn on the power transmitting coil to generate the externally-generated magnetic fields.

14. A power transmitter comprising:
a housing;
one or more proximity sensors configured to generate an output signal indicative of proximity of a medical device to the power transmitter;
a power transmitting coil configured to generate magnetic fields for transmission external to the housing; and
circuitry programmed to determine, based at least on part on the output signal, that the medical device is within range of the power transmitter and, in response, to turn on the power transmitting coil to generate the magnetic fields.

15. The power transmitter of claim 14, wherein the circuitry is further programmed to determine that an object other than the medical device is within range of the power transmitter and, in response, reduce or turn off an amount of power applied to the power transmitting coil.

16. The power transmitter of claim 14, wherein the circuitry is further programmed to determine that a battery of the medical device has a charge level lower than a threshold and, in response, to turn on the power transmitting coil.

17. The power transmitter of claim 14, wherein the output signal is a signal strength indicator.

18. A method comprising:
determining that a medical device is within a first range from the power transmitter;
applying power to a power coil of a power transmitter after the determining that the medical device is within the first range;
generating magnetic fields by the power coil to recharge a battery of the medical device;
determining that an object other than the medical device is within a second range from the power transmitter; and
reducing or turning off power applied to the power coil after the determining that the object is within the second range.

19. The method of claim 18, wherein the determining that the medical device is within the first range and the determining that the object is within the second range are both based, at least in part, on an output signal from one or more proximity sensors.

20. The method of claim 18, further comprising:
transmitting the magnetic fields towards a magnetic field guide positioned adjacent to a recharging coil positioned in the medical device.
